(19) United States
(12) Patent Application Publication
(10) Pub. No.: US 2017/0202634 A1
(43) Pub. Date: Jul. 20, 2017

(54) MEDICAL REFERENCING DEVICE, MEDICAL NAVIGATION SYSTEM AND METHOD

(71) Applicant: Aesculap AG, Tuttlingen (DE)

(72) Inventors: Stefan Gassner, Immendingen-Hattingen (DE); Steffi Altmann, Donaueschingen (DE); Markus Mueller, Albstadt (DE)

(21) Appl. No.: 15/278,174

(22) Filed: Sep. 28, 2016

Related U.S. Application Data
(63) Continuation of application No. PCT/EP2015/056393, filed on Mar. 25, 2015.

(30) Foreign Application Priority Data
Apr. 3, 2014 (DE) 10 2014 104 802

(51) Int. Cl. A61B 90/00; A61B 34/20

(52) U.S. Cl. CPC A61B 90/39; A61B 34/20; A61B 2034/2065; A61B 2090/3983; A61B 2090/3991

(57) ABSTRACT

A medical referencing device for a medical navigation system is provided, with which a movement of a body part, with the referencing device attached thereto, is trackable in space. The referencing device has a fastening device for fastening the referencing device to the body part and a referencing body which extends over a surface and is elastically stretchable in at least one direction. The referencing body is adaptable in shape to a contour of the body part and has a marking area with a pattern. The referencing body being configured such that during movement of the body part at least a section of the pattern is undeformed or substantially undeformed, and at least a section of the pattern is usable by the navigation system as reference for tracking the body part. A medical navigation system and a method for tracking a medical referencing device in space are also provided.

### CROSS-REFERENCE TO RELATED APPLICATIONS

This application is a continuation of international application number PCT/EP2015/056393 filed on Mar. 25, 2015 and claims the benefit of German application No. 10 2014 104 802.4 filed on Apr. 3, 2014, which are incorporated herein by reference in their entirety and for all purposes.

### FIELD OF THE INVENTION

The present invention relates to a medical referencing device for a medical navigation system, with which a movement of a body part, in particular, the thigh, with the referencing device attached thereto, is trackable in space.

The invention also relates to a medical navigation system for tracking a medical referencing device in space, comprising a medical referencing device, a detection device for creating images of the referencing device and providing image signals relating thereto and a data processing device for processing the image signals.

The invention furthermore relates to a method for tracking a medical referencing device in space with a navigation system.

### BACKGROUND OF THE INVENTION

Medical referencing devices, which are used, for example, in knee prosthetics to make it easier for a surgeon to implant an artificial knee joint, are known.

Conventional referencing devices comprise a marking device with marking elements arranged rigidly relative to one another (so-called “rigid body”) whose movement in space is tracked by a navigation system. To achieve as high an accuracy as possible, the marking device is screwed with a fastening device, usually a bone screw, to the femur bone or to the tibia bone and thereby fixed firmly to it. However, this results in an intervention with a not inconsiderable degree of invasiveness about which some surgeons and patients have reservations.

To reduce the invasiveness, it is known to fix rigid bodies on a sleeve placed around the femur (in this case, thigh) or the tibia (in this case, lower leg) or to mount them on a plate placed on the femur or the tibia, which is fixed with bands to the respective body part. The sleeve forms a fastening device with a support body for the marking device, which can undergo deformation from its, for example, originally planar shape and be adapted to the contour of the body part (for example, the femur or the tibia), so as to enable a positive fit on the body part.

Support bodies, which can be adhesively connected to the body part and to which the marking device is fixed, are also known.

With such noninvasive fastening devices, the problem arises in practice that movements of soft parts (skin, muscles and other non-rigid body tissue), in particular, when movement of the body part in space is to be detected, are transmitted via the support body to the marking device. The accuracy of the position data of the marking device—and hence of the body part—on which the navigation-supported operation is to be based, is thereby impaired. It would also be desirable to preoperatively and postoperatively detect patient data with a medical navigation system using a referencing device noninvasively attachable to the body part. Surgery times can thereby be reduced because data required therefore are already available preoperatively and need not first be determined intraoperatively. The success of the operation can be easily monitored postoperatively. In both cases, ionizing imaging procedures otherwise used, such as X-ray or CT, can be replaced.

An object underlying the present invention is to provide a referencing device of the kind mentioned at the outset, with which position data of the body part to which the referencing device is attached can be obtained in a manner which is gentle on the patient.

### SUMMARY OF THE INVENTION

In a first aspect of the invention, a medical referencing device is provided for a medical navigation system, with which a movement of a body part, in particular, the thigh, with the referencing device attached thereto, is trackable in space. The medical referencing device comprises a fastening device for noninvasively fastening the referencing device to the body part and a referencing body which extends over a surface and is stretchable in at least one direction of the extent. The referencing body is adaptable in its shape to the contour of the body part and has a marking area which has or forms a pattern. The referencing body is configured such that during the movement of the body part at least a section of the pattern is undeformed or substantially undeformed, and at least a section of the pattern is usable by the navigation system as reference for tracking the body part in space.

In a second aspect of the invention, a medical navigation system for tracking a medical referencing device in space comprises a medical referencing device, a detection device for creating images of the referencing device and providing image signals relating thereto and a data processing device for processing the image signals. The referencing device is a referencing device in accordance with the first aspect. The data processing device is so configured and programmed that it recognizes in signal components of the image signals originating from the marking area at least one section of the pattern and assesses whether this is undeformed or substantially undeformed as a function of time and, if so, regards this as reference for tracking the referencing device in space.

In a third aspect of the invention, a navigation system in accordance with the second aspect is used in a method tracking a medical referencing device in space. Images of the referencing device are created and image signals relating thereto provided by the detection device. The data processing device recognizes in signal components of the image signals originating from the marking area at least one section of the pattern and assesses whether this is undeformed or substantially undeformed as a function of time, and, if so, regards this as reference for tracking the referencing device in space.

### BRIEF DESCRIPTION OF THE DRAWINGS

The foregoing summary and the following description may be better understood in conjunction with the drawing figures, of which:

FIG. 1: shows a perspective illustration of a navigation system in accordance with the invention, comprising a referencing device in accordance with the invention fixed to a patient;

FIG. 2: shows a plan view of a first side of the referencing device from FIG. 1, configured as bandage;

FIG. 3: shows a plan view of a second side of the referencing device located opposite the first side;

FIG. 4: shows an enlarged illustration of a detail of the referencing device, which shows a section of a knitted fabric;

FIG. 5: shows the detail from FIG. 4 when force is applied to the knitted fabric;

FIG. 6: shows the referencing device from FIG. 1 during flection of the patient's leg;

FIG. 7: shows a plan view of a further embodiment of a referencing device;

FIG. 8: shows a plan view of a further embodiment of a referencing device; and

FIG. 9: shows a plan view of a further embodiment of a referencing device.

### DETAILED DESCRIPTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a medical referencing device for a medical navigation system, with which a movement of a body part, in particular, the thigh, with the referencing device attached thereto, is trackable in space. The medical referencing device comprises a fastening device for noninvasively fastening the referencing device to the body part and a referencing body which extends over a surface and is stretchable in at least one direction of the extent. The referencing body is adaptable in its shape to the contour of the body part and has a marking area which has or forms a pattern. The referencing body is configured such that during the movement of the body part at least a section of the pattern is undeformed or substantially undeformed, and at least a section of the pattern is usable by the navigation system as reference for tracking the body part in space.

Incorporated in the invention is the consideration that a marking device, in particular, in the form of a so-called “rigid body”, as used in conventional referencing devices, can be dispensed with by the function carried out by the marking elements of the marking device being able to be transferred to one or more other components of the referencing device. For this purpose, the present invention proposes using a deformable referencing body having a surface and being adaptable in its shape to the contour of the body part (for example, the femur or the tibia). The referencing body can be fixed by way of the fastening device of the referencing device noninvasively and, therefore, in a manner which is gentle on the patient to the body part. For this purpose, the fastening device can comprise, for example, one or more fixing elements. The referencing body is elastically stretchable in at least one direction of its extent. This allows the referencing body to be placed under tension in the state attached to the body part. Practice shows that the soft parts can thereby be compressed during the stretching of the referencing body. This allows movement of the soft parts relative to one another to be reduced. The movement induced by the body part onto the referencing body can thereby also be reduced, and so the referencing body can only execute a slight movement of its own relative to the body part. Furthermore, movements of soft parts relative to one another can be absorbed within the referencing body because it is elastically stretchable. In order that the referencing device can be tracked in space, the referencing body comprises a marking area which has or forms a pattern. The pattern is so configured that it is recognizable by the navigation system and can be regarded by the navigation system as reference for tracking the body part with the referencing device attached thereto in space. For this purpose, the referencing body is so configured that when the body part moves, at least a section of the pattern is undeformed or substantially undeformed owing to, as mentioned hereinabove, any movements of soft parts being so absorbed within the referencing body that at least a section of the pattern is not influenced thereby or only influenced thereby to an insignificant extent. Accordingly, the navigation system can recognize at least a section of the pattern as “spatially stable” and use it as valid reference. In particular, the navigation system can assume that a pattern is arranged in unchanged position relative to the femur bone when it is recognized as valid reference. Consequently, conclusions can also be drawn about the position of the femur bone in space.

The pattern is preferably a regular pattern.

The pattern can, for example, be grid-shaped, grating-shaped or net-shaped.

It proves advantageous for the referencing body to be elastically stretchable in the surface of its extent in two directions aligned transversely to each other. For example, the referencing body is elastically stretchable in a direction which, in the state attached to the body part, extends in the circumferential direction of the body part, and in a direction aligned transversely thereto, in particular, in the longitudinal direction of the body part.

The stretchabilities of the referencing body preferably differ from each other in the two directions. It may, for example, be provided that the stretchability of the referencing body in the state attached to the body part is greater along the length of the body part than in the circumferential direction of the body part.

Expediently, the marking area is integrally incorporated in the referencing body. In this case, this can be understood, in particular, as meaning that the marking area is produced from the manufacturing point of view as component of the referencing body. For example, the marking area is knitted into the referencing body, as will be explained in more detail hereinbelow, or is formed therein.

In an advantageous embodiment of the referencing device in accordance with the invention, the marking area forms the referencing body.

The marking area can completely include the pattern.

In a further advantageous embodiment, the referencing body expediently has a non-marking area adjoining the marking area, the stretchability of the non-marking area differing from that of the marking area. In this case, an area of the referencing body not including the pattern of the marking area is regarded as non-marking area. By providing different stretchabilities in at least one direction in the surface of the extent of the referencing body—preferably in two directions aligned transversely to each other—it is possible to absorb movements of soft parts specifically in the referencing body such that a movement of the marking area relative to the body part can be substantially or completely avoided. The pattern or a section thereof thereby remains so uninfluenced by the movement that it can serve as valid reference for tracking the body part in space.

The non-marking area may, for example, surround the marking area. For example, the non-marking area forms an enclosure for the marking area in the surface of the referencing body. The non-marking area can form, in particular, an edge of the referencing body, which encloses the marking area.

In a plan view, the referencing body can have a rectangular or substantially rectangular contour. For example, this is the case when the referencing body is a section or component of a bandage configured by the referencing device, as will be explained in more detail hereinbelow.

In an advantageous implementation of the referencing device, it proves expedient for the marking area, in a plan view of the surface of the referencing body, to have a rectangular or substantially rectangular contour. This is advantageous, in particular, when the non-marking area, as mentioned hereinabove, encloses the marking area and forms an edge of the referencing body.

In a different implementation of the referencing device, it is advantageous for the marking area, in a plan view of the surface of the referencing body, to have a tapered contour with two widening sections and a narrowing section arranged between these. Such a configuration is used, for example, in a referencing device in the form of a thigh bandage. The orientation of the marking area with widening section-narrowing section-widening section is preferably in the longitudinal direction of the femur.

It may be provided that the stretchability of the non-marking area in at least one direction of the extent of the referencing body is less than the stretchability of the marking area. The stretchability of the non-marking area can also be less in two directions of the extent of the referencing body aligned transversely to each other than that of the marking area. When the referencing device is applied to the thigh or to the tibia, which results in a deformation of the referencing body, the first of the two directions runs, for example, in the circumferential direction of the thigh or the tibia. The direction aligned transversely thereto runs, for example, in the longitudinal direction of the thigh or the tibia.

Preferably, the marking area and the non-marking area are integrally connected to each other, whereby the referencing body is constructionally easy to manufacture.

It is advantageous for the referencing body to be a textile material or to comprise such a textile material, preferably a knitted fabric, a warp-knitted fabric or a woven fabric, at least at the marking area, the pattern being formed by the type of weave of the textile material and/or by the structure and/or by the color of the threads used for its manufacture. This makes it possible to incorporate the pattern integrally into the marking area. In addition, it is possible to design the referencing body at the marking area elastically in a simple way from the manufacturing point of view. The type of weave of the textile material results, for example, in a surface structure and/or a coloring of the marking area, which can be interpreted by the navigation system as pattern. Thread-shaped starting material for the textile material can comprise natural and/or artificial fibers and/or elastic threads or consist thereof.

The referencing body can have a non-marking area adjoining the marking area, the marking area and the non-marking area being integrally incorporated in the textile material, and the at least one marking area and the non-marking area differing from each other in the structure of the textile material. The non-marking area—for example, the aforementioned non-marking area—and the marking area can thereby be provided with different characteristics, in particular, with different stretchabilities. This allows a simple, versatile and cost-effective manufacture of the referencing device.

The marking area and the non-marking area are preferably connected to each other by knitting, warp-knitting or weaving. This makes it possible to knit, warp-knit or weave the marking area and the non-marking area, in particular, the referencing body, in a single, uniform manufacturing method. In particular, it is not necessary to manufacture the marking area and the non-marking area separately from each other and connect them to each other in a different way, for example, by sewing.

The marking area and the non-marking area differ from each other, for example, by a color, a color pattern, a structure of the surface of the textile material, by the respective type of weave of the marking area and the non-marking area and/or by the respective structure of the threads used (in particular, the number, the density, the color and/or the stretchability of the threads).

In an implementation of the referencing device in practice, it proves expedient for the marking area to be or comprise a knitted fabric with twill weave (especially with elastic threads) or a different weave with high double-stretch elasticity (e.g. tuck weave) and/or for the non-marking area to be or comprise a knitted fabric with semi-tubular weave or a different weave with low double-stretch elasticity (e.g. right-right weave without elastic thread, Jacquard weave, Milano rib weave). A high stretchability of the marking area can, for example, be achieved by using elastically stretchable knitting threads in the stretch area.

In a different advantageous embodiment of the referencing device in accordance with the invention, it is expedient for the referencing body to be made of a rubber material, in particular, an elastomer.

In particular, in the last-mentioned advantageous embodiment, it may be provided that the pattern at the marking area is applied to the referencing body, for example, by printing, embossing, forming or adhesive bonding.

It is advantageous for the referencing device to be configured as bandage or to comprise such a bandage, which is transferable from an open released state to an applied state closed within itself, the fastening device comprising one or more fixing elements for fixing the bandage in the applied state. The referencing body can form a section of the bandage to which, for example, fixing elements of the fastening device are attached, with which the bandage is fixable in the closed, ring-shaped applied state. The fixing elements comprise, for example, a strap, a loop or an eyelet through which the strap can be passed in order to tighten the bandage or a clasp for the same purpose. Further examples of fixing elements are press-studs, a hook-and-loop fastener or an adhesive layer, for example, in order to fix the strap within itself. An advantageous implementation comprises a strap and a loop, in order that the bandage can be applied tautly to the body part, in particular, the femur, with the referencing body being stretched.

The bandage can comprise stiffening elements, which are arranged at opposite sides of the referencing body. The stiffening elements can ensure that the referencing body does not taper as a result of tautening.

The length of the bandage in the applied state is advantageously adaptable for a positive fit on the body part. For this purpose, for example, fixing elements of the fastening device in the form of a strap and a loop can interact for passage of the strap therethrough. The strap can be fixed within itself, for example, by means of a hook-and-loop fastener.

In a different advantageous implementation, it is expedient for the referencing device to be configured as tubular bandage closed within itself or to comprise such a tubular bandage closed within itself. The tubular bandage, which is formed entirely or in a section thereof by the referencing body, can be expanded, thereby undergoing stretching, and slipped over the body part. This makes it possible to fix the tubular bandage with a positive fit on the body part and to compress the body part.

The bandage is preferably a thigh bandage. At an edge, the thigh bandage can have a recess in which the kneecap or soft tissue arranged above the kneecap can engage. This makes it possible to align the thigh bandage in a defined manner relative to the body part, here the femur. A section with low deformability of the thigh bandage, for example, the aforementioned non-marking area, can be arranged at the edge of the recess. When the bandage is applied, this section can be positioned above the tendon of the quadriceps muscle. There is little movement within the referencing body above the tendon of the quadriceps muscle, and a movement within the bandage can be kept low. For this reason, the marking area is preferably arranged near the recess, in particular, above the tendon of the quadriceps muscle.

It is expedient for the referencing body to have friction coefficient increasing elements on its side facing the body part. The friction coefficient increasing elements can be arranged non-uniformly on the referencing body. Soft parts can be additionally tautened via the friction coefficient increasing elements. This proves advantageous, in particular, with one of the aforementioned bandages. The referencing device can be additionally stabilized on the body part in the area of the friction coefficient increasing elements without creating an arrest of blood circulation at the body part even when the referencing device is tautly applied. Preferably, it is possible to introduce movements of body tissue relative to one another via the friction coefficient increasing elements in a more targeted manner into the referencing body and absorb them therein, in order to reduce a transmission of movements onto the marking area and the pattern.

It may be provided that a section of the marking area is free of friction coefficient increasing elements.

The friction coefficient increasing elements are preferably made of a silicone material or can comprise such a silicone material. The friction coefficient increasing elements may have different shapes and/or different sizes. For example, the friction coefficient increasing elements are point-shaped, for example, approximately the size of pin heads. At least one thread, which is worked into the aforementioned textile material, for example, knitted into a knitted fabric, can be provided as friction coefficient increasing element. The thread consists, for example, of a silicone material.

Friction coefficient increasing elements are preferably provided along at least one edge or parallel to an edge of the referencing body. For example, in a bandage in the circumferential direction of the bandage, in relation to its applied state, friction coefficient increasing elements can be arranged at the edge of the referencing body.

It is expedient for friction coefficient increasing elements to be provided at two opposite edges of the referencing body extending parallel to each other. For example, friction coefficient increasing elements are present at opposite edges of the bandage along its circumferential direction.

Alternatively or additionally, it may be provided that there are provided at one edge two sections of the referencing body which are provided with friction coefficient increasing elements and between which there is arranged a section of the referencing body which is not provided with friction coefficient increasing elements. In particular, a section of the referencing body without friction coefficient increasing elements can be provided at the edge of the aforementioned recess of a bandage.

In the last-mentioned advantageous embodiment, the friction between the body part, for example, the thigh, and the referencing body can be reduced at the section without friction coefficient increasing elements. This proves advantageous, for example, in the case of extension and flection of the leg. In the area of the section, the referencing body is thereby engaged to a lesser extent by the skin than a section provided with friction coefficient increasing elements. As a result, the section without friction coefficient increasing elements can prove so spatially stable on the thigh that the marking area with the pattern near or in the area of this section can be used for a valid reference in relation to the femur bone. The pattern can also maintain the position relative to the femur bone in the case of extension and flection of the leg and remain free of deformation, which can be detected by the navigation system.

As mentioned at the outset, the present invention further relates to a navigation system. In accordance with the invention, a medical navigation system for tracking a medical referencing device in space comprises a medical referencing device, a detection device for creating images of the referencing device and providing image signals relating thereto and a data processing device for processing the image signals. The referencing device is a referencing device of the aforementioned kind. The data processing device is so configured and programmed that it recognizes in signal components of the image signals originating from the marking area at least one section of the pattern and assesses whether this is undeformed or substantially undeformed as a function of time and, if so, regards this as reference for tracking the referencing device in space.

The advantages achievable as explained in conjunction with the referencing device in accordance with the invention can also be achieved with use of the navigation system, and so reference is to be had to the above statements. Like the referencing device, the navigation system can do without a marking device in the conventional sense in the form of a so-called “rigid body”.

The image signals are analyzed by the data processing device as to whether at least one section of the pattern, which does not change as a function of time, can be recognized. This is to be distinguished from a change as a function of time in the image signals of the section of the pattern owing to a movement of the body part in space. If the data processing device ascertains that at least one section of the pattern is undeformed or substantially undeformed, it can regard this section as reference for tracking the referencing device and, consequently, the body part in space.

The data processing device is preferably so configured and programmed that it examines the image signals with methods for color value recognition (also includes gray value recognition) and/or pattern recognition and/or scene analysis and/or finite element calculation.

It may be provided that information relating to the pattern is stored in a memory unit of the navigation system, and that the data processing device is so configured and programmed that it compares the image signals with the information and uses this as a basis for determining the signal components originating from the marking area. On the basis of the information, the data processing device can identify signal components of the marking area and recognize at least one section of the pattern as undeformed or substantially undeformed as a function of time. The position and/or orientation of the pattern in space can thereby be determined.

As mentioned at the outset, the invention relates to a method. A navigation system of the aforementioned kind is used in a method in accordance with the invention for tracking a medical referencing device in space, wherein images of the referencing device are created and image signals relating thereto provided by the detection device, and wherein the data processing device recognizes in signal components of the image signals originating from the marking area at least one section of the pattern and assesses whether this is undeformed or substantially undeformed as a function of time, and, if so, regards this as reference for tracking the referencing device in space.

Such a method has the advantages mentioned hereinabove in conjunction with the explanation of the referencing device and the navigation system, and so reference is to be had in this connection to the above statements.

Advantageous embodiments of the method result from the features of advantageous embodiments of the referencing device in accordance with the invention and the navigation system in accordance with the invention. Reference is also to be had in this connection to the above statements.

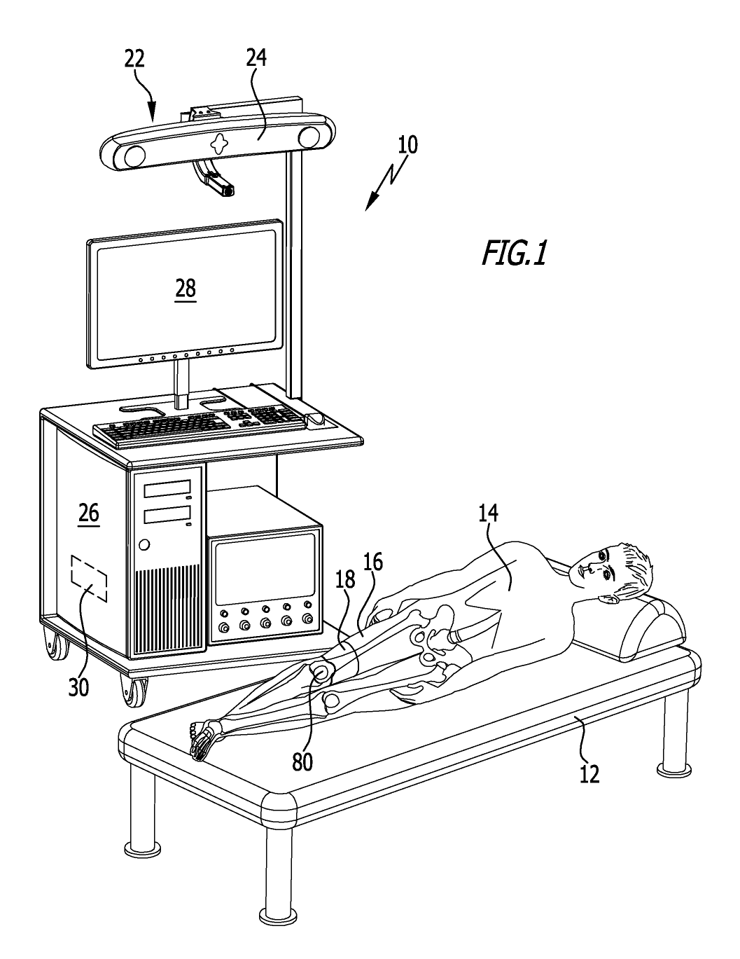

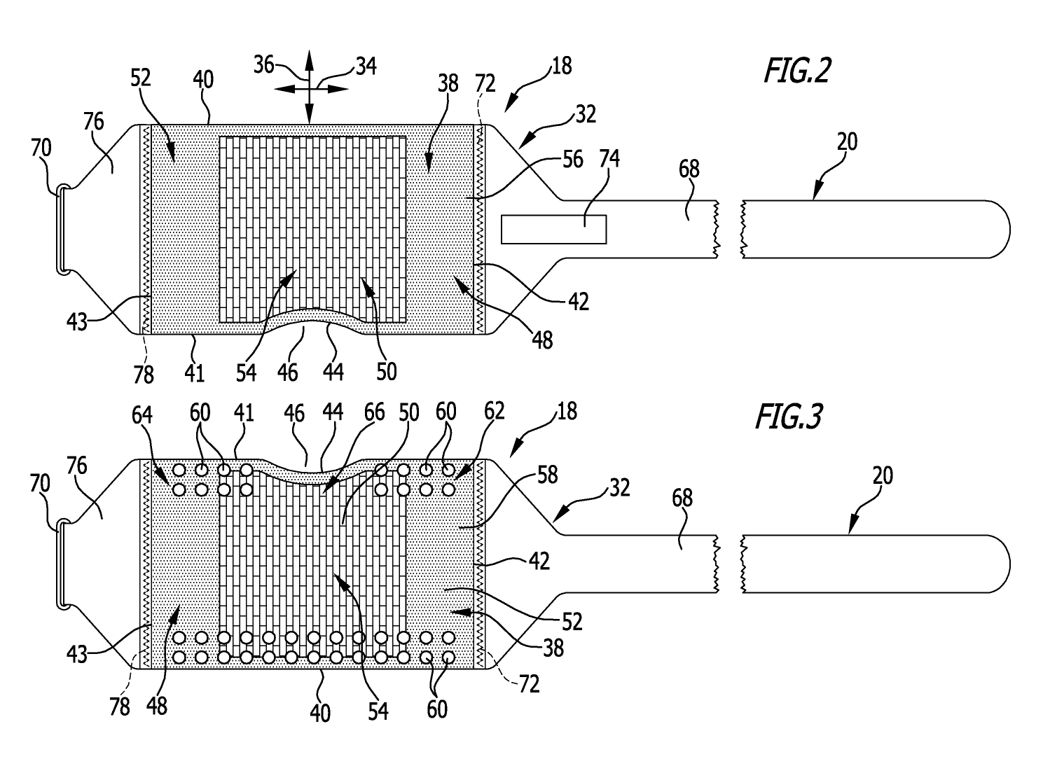

FIG. 1 shows a perspective illustration of an advantageous embodiment of a navigation system 10 in accordance with the invention and a patient 14 with a femur (in this case, thigh) 16 lying on an operating table 12. The navigation system 10 comprises an advantageous embodiment, denoted by reference numeral 18, of a referencing device in accordance with the invention. The referencing device 18 comprises a fastening device 20 (FIG. 2) with which it is noninvasively fixable to the femur 16.

When the femur 16 moves in space, the referencing device 18 also moves. A movement can be detected by a detection device 22 of the navigation system 10. For this purpose, the detection device 22 may comprise a camera. In this case, the camera is represented as stereo camera 24. It may, however, also be a different type of camera. Image signals of the detection device 22 can be transmitted to a data processing device 26. The data processing device 26 determines on the basis of the image signals how the referencing device 18 and hence the femur 16 moves in space. This will be explained in more detail hereinbelow.

The navigation system 10 further comprises a display device 28 coupled to the data processing device 26 and a memory unit 30, which is integrated in the data processing device 26.

The navigation system 10 with the referencing device 18 can be used, for example, for navigation-supported implantation of an artificial knee joint. For reasons explained hereinbelow, in particular, preoperative, intraoperative and postoperative use of the referencing device 18 is possible in a manner which is gentle on the patient.

The referencing device 18 is configured, in this case, as bandage 32, in particular, as compression bandage. The bandage 32 comprises a referencing body 38 extending over a surface in two directions of extent 34, 36 aligned transversely to each other. The referencing body 38 is changeable in shape and adaptable to the contour of the femur 16 when the bandage 32 is applied thereto. The referencing body 38, like the bandage 32 in its entirety, can thus be applied with a positive fit to the femur 16 and surround it. During use in accordance with the specifications, when the bandage 32 is applied to the femur 16, with the referencing body 38 undergoing deformation, the direction of extent 34 runs in the circumferential direction of the femur 16 and the direction of extent 36 in the longitudinal direction of the femur 16.

The referencing body 38 is of substantially rectangular shape in a plan view with two longitudinal sides 40, 41 extending parallel to each other in the direction of extent 34 and transverse sides 42, 43 in the direction of extent 36. The longitudinal side 41 has an arcuate curvature 44 in its middle section, and so a recess 46 is arranged at the edge of the referencing body 38 in the area of the curvature 44.

In this case, the referencing body 38 is a textile material 48, configured as knitted fabric. The referencing body 38 and hence the bandage 32 can therefore be simply, cost-effectively and versatilely produced from a manufacturing point of view. In this case, the textile material 48 has two areas of different weave, and owing to the different weave and the threads used, the referencing body 38 therefore has different characteristics in areas thereof.

In particular, the textile material 48 is made such that the referencing body 38 is elastically stretchable in the surface of its extent in the direction of extent 34 and in the direction of extent 36. When applying the bandage 32, the referencing body 38 can therefore be stretched in order to compress the femur 16, as explained in more detail hereinbelow.

The textile material 48 comprises two areas, namely a marking area 50 and a non-marking area 52. The marking area 50 and the non-marking area 52 are integrally incorporated in the textile material 48 in a simple way from a manufacturing point of view by knitting and in a single operation. The textile material 48 differs in its structure at the marking area 50 from its structure at the non-marking area 52. In other words, the marking area 50 and the non-marking area 52 differ from each other in the structure of the textile material 48.

In a plan view of the referencing body 38, the marking area 50 has a substantially rectangular shape. The marking area 50 is located at the center of the referencing body 38 and is surrounded by the non-marking area 52. The non-marking area 52 encloses the marking area 50 and forms an edge for it. Furthermore, the non-marking area 52 forms the longitudinal sides 40, 41 and the transverse sides 42, 43.

In this case, the marking area 50 is a knitted fabric with high double-stretch elasticity, for example, with twill weave, which, in particular, has elastic knitting threads. The non-marking area 52 is a knitted fabric with semi-tubular weave with threads of lower elasticity. As a result, the marking area 50 has in both directions of extent 34, 36 (and in a direction resulting from a superimposition of the directions of extent 34, 36 and respectively aligned at an angle to these in the surface of the referencing body 38) a greater stretchability than the non-marking area 52. The stretchability of the marking area 50 is greater in the direction of extent 36 than in the direction of extent 34.

The areas 50, 52 may preferably differ not only with respect to the type of weave and the elasticity of the threads used. In particular, the areas 50, 52 can also be made of threads of a different color. Image signals of the areas 50 and 52 can thereby be easily separated from one another by the data processing device 26.

In this case, the marking area 50 is knitted such that its type of weave and/or the structure and/or, in particular, the color of the threads used for the manufacture form a pattern 54. The pattern 54 is thereby integrally incorporated in the referencing body 38 by the knitting operation. In a state in which it is laid out flat, the pattern 54 is uniform throughout the entire marking area 50. The pattern 54 is preferably regular.

If the marking area is distorted, there may be sections thereof in which the pattern is distorted and appears different. In contrast, at least one section can be provided in which the pattern 54 appears undistorted or substantially undistorted. In this case, this is regarded as “undeformed” or “substantially undeformed” pattern. This also applies during use of the bandage 32 in accordance with the specifications, during which the referencing body 38 becomes deformed when applied to the femur 16 and assumes its contour. At least sections of the pattern 54 can be free or substantially free of deformation and recognizable for the navigation system 10 in a way as if the referencing body 38 were to assume a state in which it is laid out flat.

The non-marking area 52 can also have a pattern owing to the type of weave, the structure and the color of the threads used, with this pattern differing, in this case, from the pattern 54 of the marking area 50. For example, the different types of weave of the textile material 48 at the areas 50, 52 already result in a different patterning thereof owing to different surface structures.

FIGS. 2 and 3 show schematically the bandage 32 in a plan view of a first side (upper side 56 hereinbelow) and a second side (underside 58 hereinbelow). During use of the bandage 32 in accordance with the specifications, the underside 58 faces the femur 16 and the upper side 56 faces away from the femur 16.

On the underside 58, the referencing body 38 has a plurality of friction coefficient increasing elements 60 (hereinbelow, for the purpose of simplification, friction elements 60). In this case, the friction elements 60 are point-shaped and, for example, approximately the size of a pin head. The friction elements 60 are preferably made of a silicone material.

The referencing body 38 comprises friction elements 60 at the edge which has the longitudinal side 40. The friction elements 60 extend in a double row parallel to the longitudinal side 40 and are uniformly spaced from one another. The friction elements 60 extend over almost the entire length of the referencing body 38 and are arranged on both areas 50, 52.

At the opposite edge forming the longitudinal side 41, the referencing body 38 comprises a first section 62 and a second section 64, each with friction elements 60, and a section 66 lying between these, which is free of friction elements 60. The section 62 is arranged in the direction of extent 34 between the recess 46 and the transverse side 42, and the section 64 between the recess 46 and the transverse side 43. The section 66 extends somewhat beyond the length of the recess 46 at the center of the referencing body 38. At each section 62, 64, the friction elements 60 extend in a double row parallel to the longitudinal side 41 and are respectively arranged equidistantly from one another on the referencing body 38.

The friction elements 60 make it possible to increase the friction between the bandage 32 and the femur 16 locally. In the area of the section 66, the friction is specifically kept low.

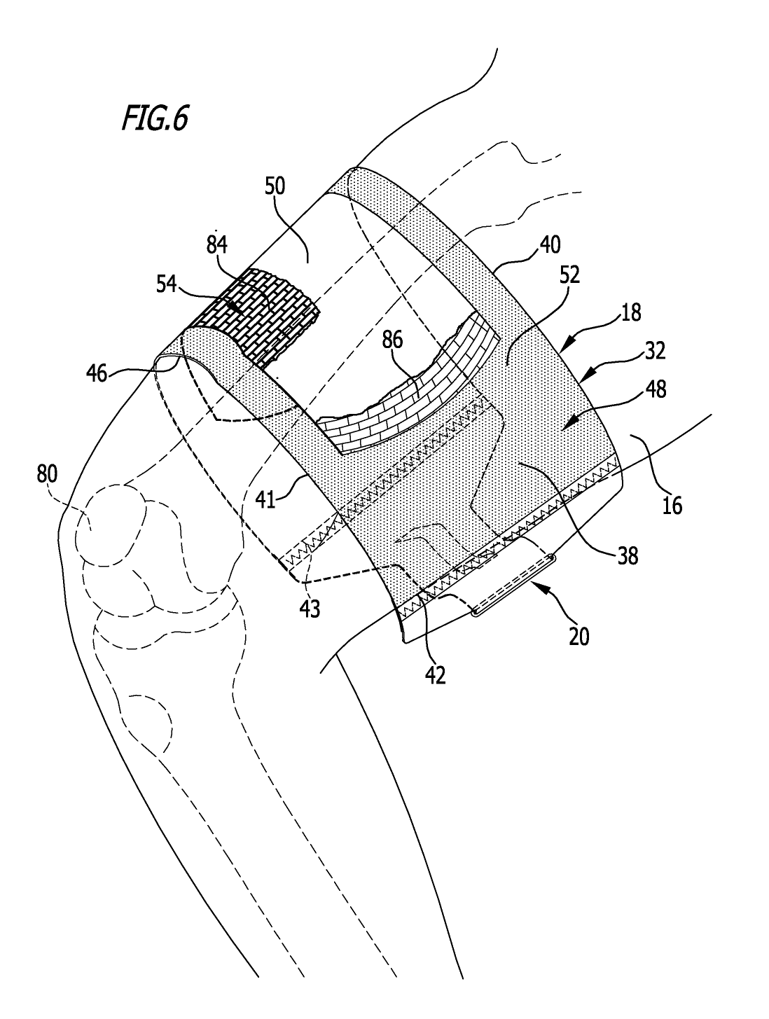

The bandage 32 is a bandage which is adjustable in size. From an open released state shown in FIGS. 2 and 3, it is transferable to an applied state in which the bandage is closed within itself (FIGS. 1 and 6). For this purpose, the fastening device 20 has fixing elements, namely a strap 68 and an eyelet 70 interacting therewith. The strap 68 is fixed to the transverse side 42 via a stiffening element 72 and extends along the direction of extent 34.

The stiffening element 72 comprises, for example, a fabric tunnel with a helical spring or strip made, for example, of plastic, arranged therein. Near the transverse side 42, there is provided a fixing element of the fastening device 20 in the form of a hook-and-loop fastener 74, to which the strap 68 can be adherently attached in a manner closed within itself.

The eyelet 70 is attached to a side of a fixing element of the fastening device 20 in the form of a holding section 76. The holding section 76 is connected at the opposite side of the eyelet 70 via a stiffening element 78 at the transverse side 43 to the referencing body 38. The stiffening element 78 corresponds in form and function to the stiffening element 72.

To transfer the bandage 32 to the applied state on the femur 16, the underside 58 is noninvasively positioned on the skin of the patient in a manner which is gentle on the patient. The bandage 32 is preferably stretched in the direction of extent 34, counter to the elasticity of the areas 50, 52. The bandage 32 is preferably positioned on the femur 16 such that soft tissue above the kneecap 80 engages the recess 46 (with the leg stretched). When the soft tissue above the kneecap 80 engages the recess 46, the marking area 50, in particular, in the area of the section 66 free of friction elements 60, is arranged above the tendon of the quadriceps muscle of the femur 16.

In the applied state, the bandage 32 is fixed by the strap 68 being guided through the eyelet 70, with the bandage 32 being tautened, and attached within itself to the hook-and-loop fastener 74. The stiffening elements 72, 78 ensure that the bandage 32 maintains its shape in the direction of extent 34 and the referencing body 38 does not taper due to the tautening.

The bandage 32 and hence the referencing device 18 are noninvasively fixable to the femur 16 in a patient-friendly and user-friendly way. In the applied state, the bandage 32 compresses the femur 16. Movements of soft tissue relative to one another and relative to the femur bone are thereby restricted. Consequently, the ability of the bandage 32 to move relative to the femur 16 and, in particular, to the femur bone is also reduced.

Also, relative movements of soft tissue which do occur can be substantially absorbed within the referencing body 38. For this purpose, the manufacture of the referencing body 38 as knitted fabric 58 with the areas 50, 52, which differ with respect to their stretchability in at least one of the directions of extent 34, 36, proves particularly advantageous. The areas 50, 52 are knitted such that movements within the femur 16 are to a large extent absorbed by the referencing body 38. In particular, at the marking area 50 at the center thereof (in relation to the direction of extent 34), which comprises the section 66 free of friction elements 60, particularly little movement occurs within the referencing body 38.

The friction elements 60 and their arrangement on the referencing body 38 improve by way of the specific compression of the femur 16 the capacity of the bandage 32 to specifically introduce movements into the referencing body 38.

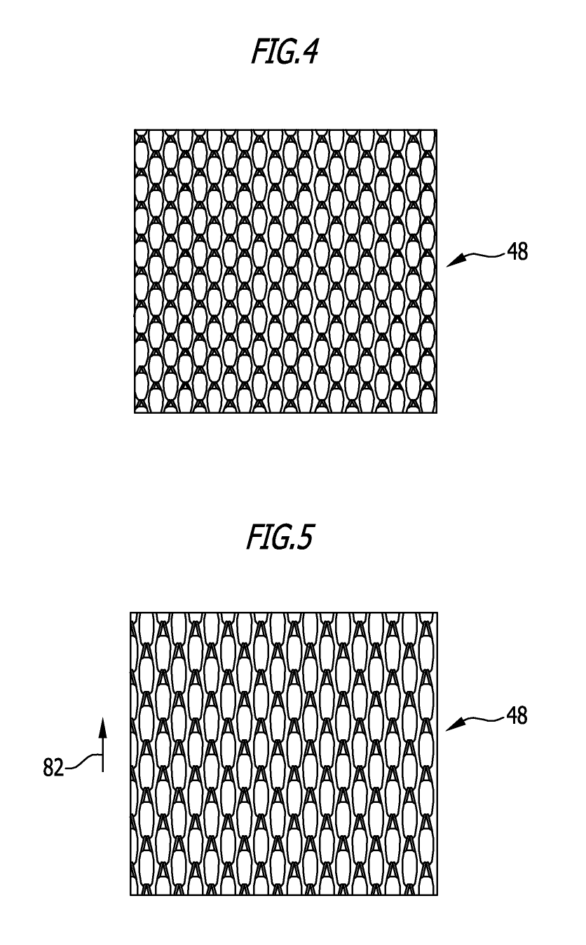

FIGS. 4 and 5 show by way of example how the textile material 48 can become deformed under the action of a force symbolized by an arrow 82. Such a force can be exerted by the femur 16 on the textile material 48 when relative movements of soft tissue occur, in particular, during movement of the femur 16 in space, flection or extension of the leg. From the example of FIGS. 4 and 5 it is clear that the textile material 48 can then become deformed with the surface structure undergoing change. This may result, in particular, in a deformation of the pattern 54 at the marking area 50. Conversely, a deformation of the pattern 54 may not occur in the absence of the force 82.

A force deforming the pattern 54 can be kept low, in particular, at the marking area 50 in the area of the section 66. As there are no friction elements 60 present at the section 66, the friction between the femur 16 and the bandage 32 is kept low locally. For example, the skin at the section 66 rubs only to a slight extent against the referencing body 38 during extension and flection of the leg, and so the referencing body 38 is not engaged or only engaged to a slight extent by a soft part displacement.

If the kneecap 80 or soft tissue above the kneecap 80 is arranged in the area of the recess 46, and the section 66 above the tendon of the quadriceps muscle, the force on the marking area 50 is low. The movement of the marking area 50 and hence the deformation of the pattern 54 is also low in this area. This is also a consequence of the relatively low movability of the tendon of the quadriceps muscle relative to other soft tissue and other muscles in the femur 16.

FIG. 6 symbolizes by way of example how during flection of the leg, consequently, a section 84 of the pattern 54, which is arranged at the section 66, is not subjected or only subjected to a slight extent to a deforming force. As a result, in the area of the section 84, the pattern 54 is not deformed or only deformed to a slight extent. The section 84 can thereby form a valid reference whose position relative to the femur bone can be maintained during a movement of the leg in space, an extension and a flection of the leg.

Whereas in the illustration, by way of example, of FIG. 6, the pattern 54 at the section 84 is undeformed and can be used as valid reference, the pattern 54 at a section 86 is deformed. For example, the section 86 is arranged at the edge of the marking area 50, extending parallel to the transverse side 42 and facing the non-marking area 52. Owing to the introduction of the deformation forces 82 into the marking area 50, the marking area 50 undergoes such stretching there that the pattern 54 becomes distorted and differs from a shape in the state in which the bandage 32 is laid out flat.

During use of the navigation system 10, the detection device 22 creates images of the referencing device 18 and provides the data processing device 26 with image signals relating thereto. The data processing device 26 is so configured and programmed that it determines signal components originating from the marking area 50. This can occur, for example, on account of the colored configuration of the marking area 50. For example, signal amounts of the non-marking area 52 are separated owing to the different structure of the textile material 46, as mentioned hereinabove, from signal amounts of the marking area 50.

With methods for color value recognition and/or pattern recognition and/or scene analysis and/or finite element calculation, the data processing device 26 can recognize whether at least one section of the pattern 54 is undeformed or substantially undeformed as a function of time. This does not relate to the images of the referencing device, which change during a movement, but to the shape of the pattern 54 at successive points in time between which the femur 16 is moved with the referencing device 18 in space. In this case, the data processing device 26 can, for example, recognize the section 84 as substantially undeformed as a function of time. As a result, the data processing device 26 can regard the section 84 as valid reference for tracking the referencing device 18 and hence the femur 16 in space. This makes it possible to also draw conclusions about the position and/or orientation of the femur bone in space.

Information relating to the pattern 54 can be stored in the memory unit 30. For example, the information includes statements about the surface structure of the marking area 50, a color and/or a color pattern. In the analysis of the image signals, the data processing device 26 can revert to the information stored in the memory unit 30, compare the image signals with the information and use the information as a basis for determining the signal components originating from the marking area 50.

The navigation system 10 and the referencing device 18 offer the great advantage that a noninvasive reference can be provided on the patient 14 preoperatively, intraoperatively and postoperatively. In particular, the navigation system 10 does without a medical marking device in the form of a so-called “rigid body”, which is usually used in medical navigation. This proves to be not only more gentle on the patient, but also more user-friendly for the surgeon.

Further advantageous embodiments of a referencing device in accordance with the invention will be explained hereinbelow. The respective referencing device can be used instead of the referencing device 18 or together with it in the navigation system 10. The functions, modes of operation and advantages mentioned hereinabove in conjunction with the referencing device 18 can also be achieved with the referencing devices mentioned hereinbelow, and so reference is to be had to the above statements.

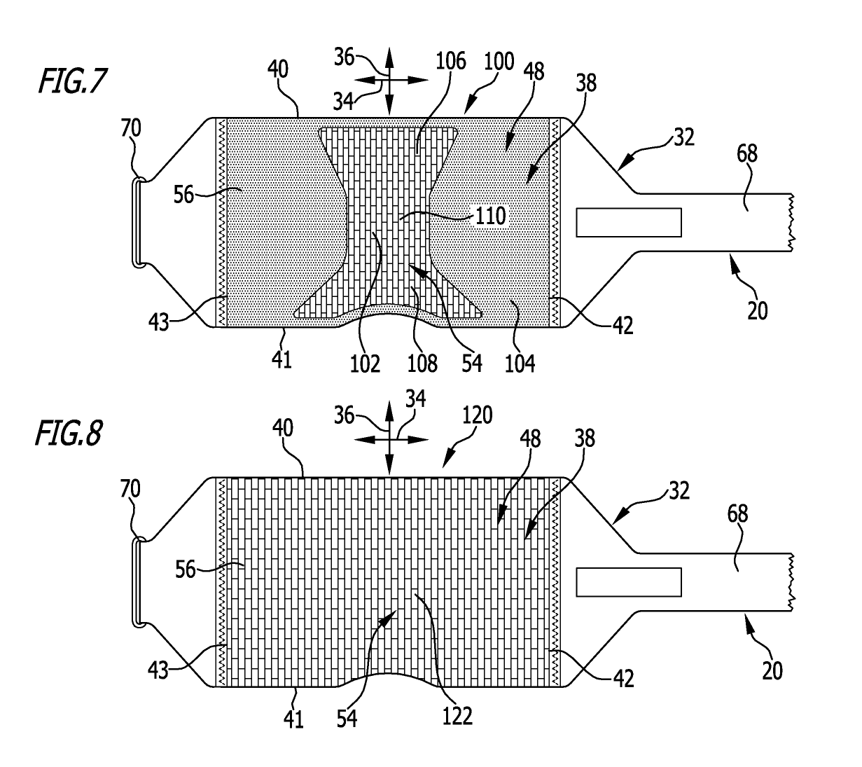

In a referencing device 100 shown in FIG. 7, the referencing body 38 is also configured as textile material 48, in particular, as knitted fabric. The referencing device 100 comprises a marking area 102 corresponding to the marking area 50 and a non-marking area 104 corresponding to the non-marking area 52. These are integrally incorporated in the textile material 48 and differ from each other with respect to the structure thereof. The marking area 102 forms the pattern 54.

The marking area 102 has, in a plan view of the referencing body 38, a tapered contour with a widening section 106 and a widening section 108 facing the longitudinal sides 40 and 41, respectively. The widening sections 106, 108 are connected to each other by a narrowing section 110. The non-marking area 104 surrounds the marking area 102 and forms an edge for it, as is the case with the referencing device 18.

A referencing device 120 shown in FIG. 8 comprises a referencing body 38 configured as textile material 48. A marking area 122 is provided, which forms the referencing body 38. A non-marking area is not provided. The marking area 122 forms the pattern 54.

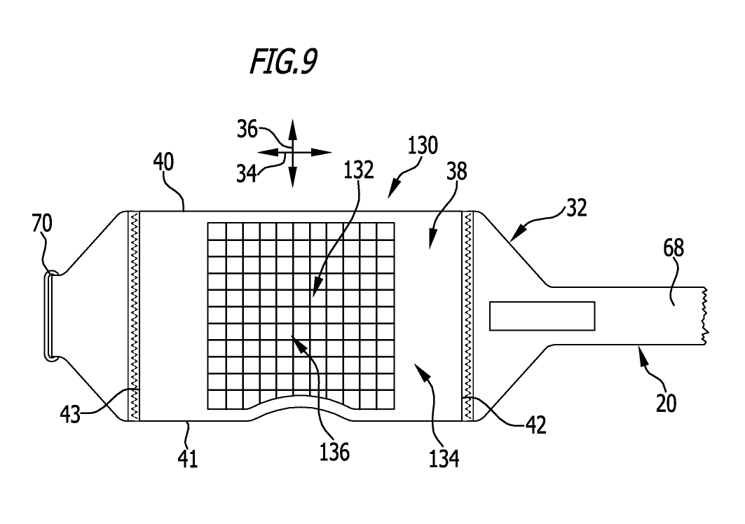

FIG. 9 shows, in a plan view, a referencing device 130 whose referencing body is made of a rubber material, in particular, an elastomer. The referencing body 38 is elastically stretchable in the directions of extent 34, 36, the stretchability being greater in the direction of extent 34 than in the direction of extent 36.

The referencing body 38 has a marking area 132, which is surrounded by a non-marking area 134. The areas 132, 134 are integrally incorporated in the referencing body 38. The shape of the areas 132, 134 and their arrangement on the referencing body 38 correspond to those of the areas 50, 52 in the referencing device 18.

A pattern 136 is provided at the marking area 132. The pattern 136 is, for example, integrally incorporated in the marking area 132, for example, by forming. Embossing or printing of the marking area 132 are also conceivable. The pattern 136 is preferably regular. The pattern 136 preferably has a two-dimensionally grid-shaped or net-shaped configuration.

With the referencing device 130, too, movements of soft tissue relative to one another can be introduced into the referencing body 38 such that at least a section of the pattern 136 remains undeformed or substantially undeformed. In the way explained hereinabove, the data processing device 26 can recognize this section and regard it as valid reference for tracking the referencing device 130 in space.

For targeted introduction of forces into the respective referencing body 38, the referencing devices 100, 120 and 130 also have friction elements 60 on the respective underside 58, as was explained with the example of the referencing device 18.

What is claimed is:

1. Medical referencing device for a medical navigation system, with which a movement of a body part, with the referencing device attached thereto, is trackable in space, the medical referencing device comprising:
a fastening device for noninvasively fastening the referencing device to the body part, and
a referencing body which extends over a surface and is elastically stretchable in at least one direction of extent, the referencing body being adaptable in shape to a contour of the body part and having a marking area which has or forms a pattern,
the referencing body being configured such that during the movement of the body part at least a section of the pattern is undeformed or substantially undeformed, and at least a section of the pattern being usable by the navigation system as a reference for tracking the body part in space.

2. Referencing device in accordance with claim 1, wherein the pattern is a regular pattern.

3. Referencing device in accordance with claim 1, wherein the pattern is grid-shaped, grating-shaped or net-shaped.

4. Referencing device in accordance with claim 1, wherein the referencing body is elastically stretchable in the surface of its extent in two directions aligned transversely to each other.

5. Referencing device in accordance with claim 4, wherein the stretchabilities of the referencing body differ from each other in the two directions.

6. Referencing device in accordance with claim 1, wherein the marking area is integrally incorporated in the referencing body.

7. Referencing device in accordance with claim 1, wherein the marking area forms the referencing body.

8. Referencing device in accordance with claim 1, wherein the reference body has a non-marking area adjoining the marking area, the stretchability of the non-marking area differing from that of the marking area.

9. Referencing device in accordance with claim 8, wherein the non-marking area surrounds the marking area.

10. Referencing device in accordance with claim 8, wherein the marking area and the non-marking area are integrally connected to each other.

11. Referencing device in accordance with claim 1, wherein the referencing body is a textile material or comprises such a textile material at least at the marking area, the pattern being formed by at least one of a type of weave of the textile material, a structure of the threads used for its manufacture and a color of the threads used for its manufacture.

12. Referencing device in accordance with claim 11, wherein the textile material is a knitted fabric, a warp-knitted fabric or a woven fabric.

13. Referencing device in accordance with claim 11, wherein the referencing body has a non-marking area adjoining the marking area, the marking area and the non-marking area being integrally incorporated in the textile material, and wherein the marking area and the non-marking area differ from each other in a structure of the textile material.

14. Referencing device in accordance with claim 13, wherein the marking area and the non-marking area differ from each other by at least one of a color, a color pattern, a structure of the surface of the textile material, a respective type of weave of the marking area and the non-marking area, and the respective structure of the threads used.

15. Referencing device in accordance with claim 1, wherein the referencing body is made of a rubber material, in particular, an elastomer.

16. Referencing device in accordance with claim 1, wherein the pattern at the marking area is applied to the referencing body by printing, embossing, forming or adhesive bonding.

17. Referencing device in accordance with claim 1, wherein the referencing device is configured as bandage or comprises a bandage, which is transferable from an open released state to an applied state closed within itself, the fastening device comprising one or more fixing elements for fixing the bandage in the applied state.

18. Referencing device in accordance with claim 17, wherein the fastening device is formed such that a length of the bandage is adaptable in the applied state for a positive fit on the body part.

19. Referencing device in accordance with claim 1, wherein the referencing body has friction coefficient increasing elements on a side of the referencing body facing the body part.

20. Medical navigation system for tracking a medical referencing device in space, comprising:
a medical referencing device,
a detection device for creating images of the referencing device and providing image signals relating thereto, and
a data processing device for processing the image signals,
wherein the medical referencing device comprises:
a fastening device for noninvasively fastening the referencing device to the body part, and
a referencing body which extends over a surface and is elastically stretchable in at least one direction of extent, the referencing body being adaptable in shape to a contour of the body part and having a marking area which has or forms a pattern,
the referencing body being configured such that during the movement of the body part at least a section of the pattern is undeformed or substantially undeformed, and at least a section of the pattern being usable by the navigation system as a reference for tracking the body part in space, and
wherein the data processing device is configured and programmed such that it recognizes in signal components of the image signals originating from the marking area at least one section of the pattern and assesses whether this is undeformed or substantially undeformed as a function of time and, if so, regards this as a reference for tracking the referencing device in space.

21. Method for tracking a medical referencing device in space with a navigation system, comprising:
providing a referencing device comprising:
a fastening device for noninvasively fastening the referencing device to the body part, and
a referencing body which extends over a surface and is elastically stretchable in at least one direction of extent, the referencing body being adaptable in shape to a contour of the body part and having a marking area which has or forms a pattern,
the referencing body being configured such that during the movement of the body part at least a section of the pattern is undeformed or substantially undeformed, and at least a section of the pattern being usable by the navigation system as a reference for tracking the body part in space,
creating images of the referencing device by a detection device,
providing image signals relating to the image from the detection device to a data processing device, and
recognizing by the data processing device in signal components of the image signals originating from the marking area at least one section of the pattern and assessing whether this is undeformed or substantially undeformed as a function of time and, if so, regarding this as reference for tracking the referencing device in space.
